(19) United States
(12) Patent Application Publication
(10) Pub. No.: US 2023/0036636 A1
(43) Pub. Date: Feb. 2, 2023

(54) Thermal Imaging Device Performing Image Analysis To Facilitate Early Detection Of Distal Extremity Altered Perfusion States

(71) Applicant: Holographic Humanity, LLC, Pinecrest, FL (US)

(72) Inventor: Robert L. Hannan, Pinecrest, FL (US)

(73) Assignee: Holographic Humanity, LLC, Pinecrest, FL (US)

(21) Appl. No.: 17/813,905

(22) Filed: Jul. 20, 2022

Related U.S. Application Data
(60) Provisional application No. 63/224,257, filed on Jul. 21, 2021.

(51) Int. Cl. A61B 5/026; H04N 5/33; A61B 5/01; A61B 5/00; G16H 30/40; G16H 10/60

(52) U.S. Cl. CPC A61B 5/0261; H04N 5/33; A61B 5/015; A61B 5/6898; G16H 30/40; G16H 10/60

(57) ABSTRACT

A thermal imaging extremity abnormal perfusion detector system includes a computer processor configured to receive, analyze and store thermal images and a thermal imaging camera communicatively coupled to the processor, and configured to take at least one of photograph and video thermal images and output the thermal images to the processor. The camera is configured to be secured adjacent a patient workspace that is shaped to contain a patient; and points the thermal imaging camera at the workspace such that, responsive to taking at least one thermal image, the at least one thermal image contains the patient who is placed within the workspace. The computer processor is configured to analyze the at least one thermal image and determine from the at least one thermal image a difference in thermal states indicating altered perfusion in an extremity of the patient.

### CROSS-REFERENCE TO RELATED APPLICATIONS

This application claims the priority, under 35 U.S.C. § 119, of copending U.S. Provisional Patent Application No. 63/224,257, filed Jul. 21, 2021; the prior application is herewith incorporated by reference herein in its entirety.

### STATEMENT REGARDING FEDERALLY SPONSORED RESEARCH OR DEVELOPMENT

Not Applicable

### FIELD OF THE INVENTION

The present systems, apparatuses, and methods lie in the field of abnormal extremity perfusion detection. The present disclosure relates to imaging devices and methods that utilize digital image processing and may include artificial intelligence to analyze measurements and facilitate early detection of distal extremity altered perfusion states. The imaging devices can take advantage of the entire electromagnetic spectrum, including radio, microwave, thermal, visible, ultraviolet, sonography, computed tomography, magnetic resonance imaging, x-ray, and/or gamma rays.

### BACKGROUND OF THE INVENTION

Assessment of skin temperature (and, therefore, vascular perfusion) is an essential and integral part of every physical examination. Performed routinely with every physical examination, assessment of vascular perfusion is undertaken at regular intervals (e.g., two to four hours) in patients on an elective basis and urgently/emergently if an acute problem is suspected. Decreased skin perfusion (lower temperature) may be associated with low cardiac output states (both extremities, either arms or legs) or with acute vascular occlusion from a blockage (perhaps from an embolus, for example) that effects only one extremity. Increased extremity perfusion may be associated with sepsis and early signs of sepsis.

Despite the importance of assessing extremity perfusion, the methodology is extraordinary crude and unchanged since the early days of medicine. The caregiver touches the patient, typically with their fingertips or the back of their hand, and then categorizes the temperature and its perfusion equivalent:

| | | | | |
|---|---|---|---|---|
| | cool and | | | |
| warm and | moderately | cold and | | |
| well perused | perfused | clammy | threatened | ischemic. |

Obviously, this determination is completely subjective. There are other problems with this methodology. The measurement depends on the observer's own skin temperature and perfusion, and there is inter-observer variability based on the observer's talent and experience, as well as the observer's physical condition—tired, distracted, and/or at the end of a long shift, for example. Further, there always exists the possibility of stochastic and systematic human error.

Thermal imaging cameras digitally measure temperature in the infrared spectrum, essentially measuring heat. They take a digital picture or video in the infrared spectrum just as regular cameras take a picture or a video in the visual spectrum. Thermal imaging cameras are accurate—they are able to digitally detect differences in temperature of as little as 0.05 degrees Fahrenheit. Temperature may be represented on a sensor display with a variety of appearances, including grey scale or vivid colors representing different temperatures. An example of decreased skin perfusion in the fingers of the left hand 1 of a patient is shown in the infrared photograph of FIG. 1. There, the lighter (golden) color indicates relative warmth and the darker (purple) color indicates relative coolness. It is apparent in the infrared photo of FIG. 1 that the patient is experiencing decreased skin perfusion in the left hand 1.

As in any visual spectrum digital camera, the infrared image of a thermal imaging camera may be digitally recorded as a still picture or a movie or both. The imaging also can be live-streamed. Because this information is digital, the detected information can be analyzed (manually or automatically) as any digital data can for changes over time in a specific location. This data may be obtained and analyzed at any interval desired, continuously in real time or retrospectively, and streamed live or retrospectively to distant locations for further analysis, and/or it can be analyzed in real-time by live-stream. It may be compared to previous determinations on the same patient or known heuristic trends with machine learning or artificial intelligence. Thermal and other imaging techniques have not been used to date to automatically detect and identify to caregivers abnormal (increased or reduced) skin perfusion indicating pathological states.

Thus, a need exists to overcome the problems with the prior art systems, designs, and processes as discussed above.

### SUMMARY OF THE INVENTION

The systems, apparatuses, and methods described provide imaging devices and methods that utilize digital image processing with or without artificial intelligence to analyze measurements and facilitate early detection of abnormal distal extremity perfusion states that overcome the herein afore-mentioned disadvantages of the heretofore-known devices and methods of this general type and that provide such features with a system, device, and method for automatically detecting, identifying, and reporting to caregivers reducing or reduced skin perfusion.

Electronic imaging technology is available throughout the electromagnetic spectrum, including, for example, radio, microwave, thermal, visible, ultraviolet, sonography, computed tomography, magnetic resonance imaging, x-ray, and gamma ray. While exemplary embodiments herein are described with respect to use of the infrared spectrum in thermal imaging, any of the other possible electronic imaging techniques using different ranges of the spectrum are interchangeable or additional (combined in any manner or number). Therefore, mention of the infrared spectrum and/or thermal imaging herein is to be considered as merely an example—all other possible imaging techniques are equally applicable without specific repetitive mention thereto.

One particularly inexpensive and ubiquitous method of determining perfusion is through temperature, which, logically, first points to the infrared spectrum and use of thermal imaging. While infrared or thermal imaging is a beneficial and easy way to obtain temperature measurement, other aspects of the electromagnetic spectrum might be more beneficial for determining perfusion depending on what results are desired to be obtained and/or how fast and/or accurate the measurement(s) needs to be. Therefore, single- or multi-spectrum imaging devices are applicable for use in each of the exemplary embodiments described herein, but are not listed or explained for reasons of brevity and removing redundancy.

The systems, apparatuses, and methods read digitized data, analyzes it utilizing digital imagery processing (and, optionally, artificial intelligence (AI) driven software) based on one or more of the location of changes on the extremity and trend of the temperature on that extremity, and compares the extremity to one or more of another extremity, the history of the patient, and/or known altered perfusion states and each of their characteristic image characteristics, and trends it/them over time. Conventional digital image processing renders the image into a digital format, artificial intelligence or machine learning software examines that data and, using the known history of the patient and characteristics of known altered perfusion states, determines if the patient is experiencing a pathological state. A simple example of such an examination (whether through process or artificial intelligence, can be illustrated with regard to FIG. 1. When investigation of a patient begins, the user determines a baseline acceptable reading, whether with a preset input value or through direct measurement of the patient (e.g., a temperature detected at location on the patient, for example, the right hand). In this example, the right hand has a measurement (a corresponding temperature and color) indicating that profusion is nominal. Thereafter, the program/AI is run to compare (e.g., periodically or continually) that measurement to a current measurement. When the measurement indicates a change to a different value, again, corresponding to a different temperature (e.g., higher) and color, then a warning is generated. For example, the temperature/color shown on the left hand in FIG. 1 is an abnormal measurement. When the comparison/determination is positive, the systems and methods automatically alarm to alert caregivers, locally or remotely or both to advise the caregivers that profusion is abnormal. This analysis provides results that indicate different causes of temperature variation, including, for example, impeding sepsis (increase in temperature bilaterally), bilateral decreasing temperature (decreasing cardiac output), or unilateral decreasing temperature (arterial of venous occlusion, from an embolus or vascular trauma, for example). The digital analysis is far more accurate than manual determination of extremity perfusion (e.g., temperature readings are accurate to 0.05 degrees Fahrenheit) and is timelier than intermittent core temperature determinations (measured in tenths of a second or a second (or even milliseconds)). The algorithm flows form data acquisition to straightforward digitization in three-dimensional space including trending and thenceforth to analysis with previous patient trends or previously identified trends and patterns of temperature with machine learning and or artificial intelligence. Appropriate alarms are triggered for concerning changes, both static and dynamic.

The analyzed data is integrated into the patient's electronic medical record in real time and drives real time alarms for appropriate warnings, for an early indication of vascular occlusion or decreased vascular perfusion caused by decreased cardiac output or of increased temperature because of sepsis. The system is completely portable and is as suitable for use out of the hospital (at home or at other facilities such as nursing homes) as well as in the hospital. The data is easily monitored remotely as well as locally with, for example, the built-in wireless connectivity to the internet cloud and/or through short-distance, direct wireless communication (e.g., Bluetooth®).

The systems, apparatuses, and methods dramatically improve accuracy, reliability, reproducibility, and timeliness (instantaneous instead of episodic) of the extremity perfusion data. The importance of extremity perfusion data is demonstrated by the necessity to document it frequently in the patient's medical record to meet the standard of care, independent of the method used. The accuracy, simplicity, and timeliness of these new devices, systems, and methods described and shown herein provide important early warning of pathological states far before they are detectable by the crude methods of human physical examination, which is performed infrequently as well as being notoriously inaccurate and difficult to calibrate and reproduce.

In an exemplary process for carrying out the method, a patient is admitted to the ICU, or another caregiver location (even to a suitably equipped home environment). One or more cameras are placed at a foot of the bed (e.g., thermal camera(s)) to observe, for example, both legs of the patient. If desired, markers are placed on one or both legs as indicators of anatomy (knee, toe, etc.: referred to herein as “registering locations”). If desired, the central temperature (axillary, rectal, bladder) of the patient may be also continuously monitored if necessary. The camera(s) and associated processing devices are programmed to determine the temperature of the patient's skin, the location of temperature on one or more extremities, the temperature of one extremity compared to the other extremity, and trends of the detected temperatures. Digital image processing software (and, optionally, artificial intelligence (AI) driven software) analyzes the detected/measured data and determines if the trend is within previously determined normal variation. The data may be compared to core temperature (either determined by a lookup table, by the user of the system, or from within the electronic medical record (EMR)) and the systems and methods determine if the comparison result indicates a sign of bilateral or unilateral extremity altered and abnormal perfusion. In an exemplary embodiment, all data is recorded in the EMR and alarms are triggered as desired and/or programmed.

With the foregoing and other objects in view, there is provided, a thermal imaging extremity altered perfusion detector system comprising a computer processor configured to receive and to at least temporarily store thermal images and a thermal imaging camera communicatively coupled to the computer processor and configured to take at least one of photograph and video thermal images and output the thermal images to the computer processor. The thermal imaging camera is configured to be secured adjacent a patient workspace that is shaped to contain a patient and to be pointed at the patient workspace such that, responsive to taking at least one thermal image, the at least one thermal image contains the patient who is placed within the workspace. The computer processor is configured to analyze the at least one thermal image and determine from the at least one thermal image a difference in thermal states indicating altered perfusion in at least one of the extremities of the patient.

With the objects in view, there is also provided a thermal imaging extremity altered perfusion detector system comprising a smartphone or similar device configured to receive and to at least temporarily store thermal images, markers configured to be placed on the extremities of the patient as registering locations, and a thermal imaging camera communicatively coupled to the smartphone/computer processor and configured to take at least one of photograph and video thermal images and output the thermal images to the smartphone, or other device. The thermal imaging camera is configured to be secured adjacent a patient workspace that is shaped to contain a patient and to be pointed at the patient workspace such that, responsive to taking at least one thermal image, the at least one thermal image contains the patient who is placed within the workspace. The smartphone/processor is configured to analyze the at least one thermal image and determine from the at least one thermal image a difference in thermal states at least one of at and adjacent the markers indicating altered (hypoperfusion or hyperfusion) in at least one of the extremities of the patient in real-time, based upon the determined difference, to communicate an indication of the altered perfusion state of the patient indicating at least one of impeding sepsis, bilateral decreasing temperature, and unilateral decreasing temperature, and to integrate the altered perfusion state into an electronic medical record of the patient.

With the objects in view, there is also provided an imaging extremity altered perfusion detector system comprising a computer processor configured to receive and to at least temporarily store electronic images and an imaging camera communicatively coupled to the computer processor and configured to take at least one of photograph and video images and output the images to the computer processor. The imaging camera is configured to be secured adjacent a patient workspace that is shaped to contain a patient and to be pointed at the patient workspace such that, responsive to taking at least one image, the at least one image contains the patient who is placed within the workspace. The computer processor is configured to analyze the at least one image and determine from the at least one image a difference in states indicating altered perfusion in at least one of the extremities of the patient.

In accordance with another feature, the computer processor is one of a smart phone and a tablet or similar portable device.

In accordance with a further feature, the thermal imaging camera is a forward-looking infrared camera.

In accordance with an added feature, the thermal imaging camera is a forward-looking infrared camera and, together with the computer processor, form a transportable IR camera system configured to acquire and to at least temporarily store the at least one thermal image.

In accordance with an additional feature, the at least one thermal image comprises an image of at least a portion of the patient and the transportable IR camera system is configured to analyze the at least one thermal image and thereby determine altered perfusion of at least one of the extremities of the patient.

In accordance with yet another feature, the transportable IR camera system is configured to perform real-time instantaneous monitoring of the at least one of the extremities.

In accordance with yet a further feature, the at least one thermal image is a video.

In accordance with yet an added feature, the transportable IR camera system is configured to acquire the at least one thermal image at least one of manually, periodically, and continually.

In accordance with yet an additional feature, the transportable IR camera system is configured to store the at least one thermal image at least one of locally and remotely.

In accordance with again another feature, the thermal imaging camera comprises a thermal imager configured to obtain thermal images in the form of at least one of stills and video.

In accordance with again a further feature, the computer processor comprises software and is configured to analyze the at least one of stills and video and to determine an altered perfusion state of the patient with the software.

In accordance with again an added feature, the computer processor is configured to analyze the at least one of stills and video in real-time.

In accordance with again an additional feature, the computer processor is a first computer processor, and which further comprises a second computer processor separate from the first computer processor and communicatively connected to the first computer processor through at least one communications link, the second computer processor being configured to analyze the at least one of stills and video, to determine an altered perfusion state of the patient, and to output the altered perfusion state.

In accordance with still another feature, the second computer processor communicates through the at least one communications link an indication of the altered perfusion state of the patient.

In accordance with still a further feature, the at least one communications link comprises the internet cloud and the second computer processor comprises at least one of a mainframe, a server, a desktop, and a laptop.

In accordance with still an added feature, the computer processor analyzes the difference in thermal states to indicate at least one of impeding sepsis, bilateral decreasing temperature, and unilateral decreasing temperature.

In accordance with still an additional feature, the indication of the altered perfusion state includes at least one of impeding sepsis, bilateral decreasing temperature, and unilateral decreasing temperature.

In accordance with another feature, there is provided an electronic medical record of the patient, the computer processor being configured to integrate the difference in thermal states into the electronic medical record in real time and to drive real time alarms for appropriate altered perfusion states.

In accordance with a further feature, there are provided markers configured to be placed on the extremities of the patient as registering locations, the computer processor being configured to determine the difference in thermal states at least one of at and adjacent the markers.

In accordance with yet a further feature, the computer processor is configured to determine data comprising at least one of a temperature of the patient's skin, a location of temperature on one or more extremities, a temperature of one extremity compared to another extremity, and trends of the detected temperatures and to analyze the determined data and to determine if a trend the determined data is within a previously determined normal variation.

In accordance with a concomitant feature, the at least one of photograph and video images are at least one of radio, microwave, thermal, visible, ultraviolet, sonography, computed tomography, magnetic resonance imaging, x-ray, and gamma ray images.

Although the systems, apparatuses, and methods are illustrated and described herein as embodied in imaging devices and methods that utilize artificial intelligence to analyze measurements and facilitate early detection of distal extremity abnormal perfusion states, it is, nevertheless, not intended to be limited to the details shown because various modifications and structural changes may be made therein without departing from the spirit of the invention and within the scope and range of equivalents of the claims. Additionally, well-known elements of exemplary embodiments will not be described in detail or will be omitted so as not to obscure the relevant details of the systems, apparatuses, and methods.

Additional advantages and other features characteristic of the systems, apparatuses, and methods will be set forth in the detailed description that follows and may be apparent from the detailed description or may be learned by practice of exemplary embodiments. Still other advantages of the systems, apparatuses, and methods may be realized by any of the instrumentalities, methods, or combinations particularly pointed out in the claims.

Other features that are considered as characteristic for the systems, apparatuses, and methods are set forth in the appended claims. As required, detailed embodiments of the systems, apparatuses, and methods are disclosed herein; however, it is to be understood that the disclosed embodiments are merely exemplary of the systems, apparatuses, and methods, which can be embodied in various forms. Therefore, specific structural and functional details disclosed herein are not to be interpreted as limiting, but merely as a basis for the claims and as a representative basis for teaching one of ordinary skill in the art to variously employ the systems, apparatuses, and methods in virtually any appropriately detailed structure. Further, the terms and phrases used herein are not intended to be limiting; but rather, to provide an understandable description of the systems, apparatuses, and methods. While the specification concludes with claims defining the systems, apparatuses, and methods of the invention that are regarded as novel, it is believed that the systems, apparatuses, and methods will be better understood from a consideration of the following description in conjunction with the drawing figures, in which like reference numerals are carried forward.

### BRIEF DESCRIPTION OF THE DRAWINGS

The accompanying figures, where like reference numerals refer to identical or functionally similar elements throughout the separate views, which are not true to scale, and which, together with the detailed description below, are incorporated in and form part of the specification, serve to illustrate further various embodiments and to explain various principles and advantages all in accordance with the systems, apparatuses, and methods. Advantages of embodiments of the systems, apparatuses, and methods will be apparent from the following detailed description of the exemplary embodiments thereof, which description should be considered in conjunction with the accompanying drawings in which:

FIG. 1 is a perspective view color photograph from a prior art thermal imaging camera of a patient who has reduced skin perfusion in a left hand;

FIG. 2 is a perspective view photograph of a prior art forward-looking infrared camera for a smartphone;

FIG. 3 is a perspective view photograph of the infrared camera of FIG. 2 attached to an exemplary embodiment of a smartphone;

FIG. 4 is a perspective view of an exemplary embodiment of the systems, processes, and methods described herein with the smartphone and the infrared camera of FIG. 3 mounted to an enclosure of a patient bed for an infant patient, the camera being pointed at the infant patient and the bed being in any of a hospital, at home, or another location:

FIG. 5 is a perspective view color photograph from the exemplary systems, processes, and methods of FIG. 4 with the infrared camera examining the infant patient who does not have reduced skin perfusion in either hand or foot extremities;

FIG. 6 is a perspective view color photograph from the exemplary systems, processes, and methods of FIG. 4 with the infrared camera of FIG. 3 of an infant patient who has reduced skin perfusion in a right foot;

FIG. 7 is a flow diagram of an exemplary embodiment of a process utilizing the transportable IR camera system; and

FIG. 8 is an exemplary embodiment of a data communication diagram utilizing the transportable IR camera system.

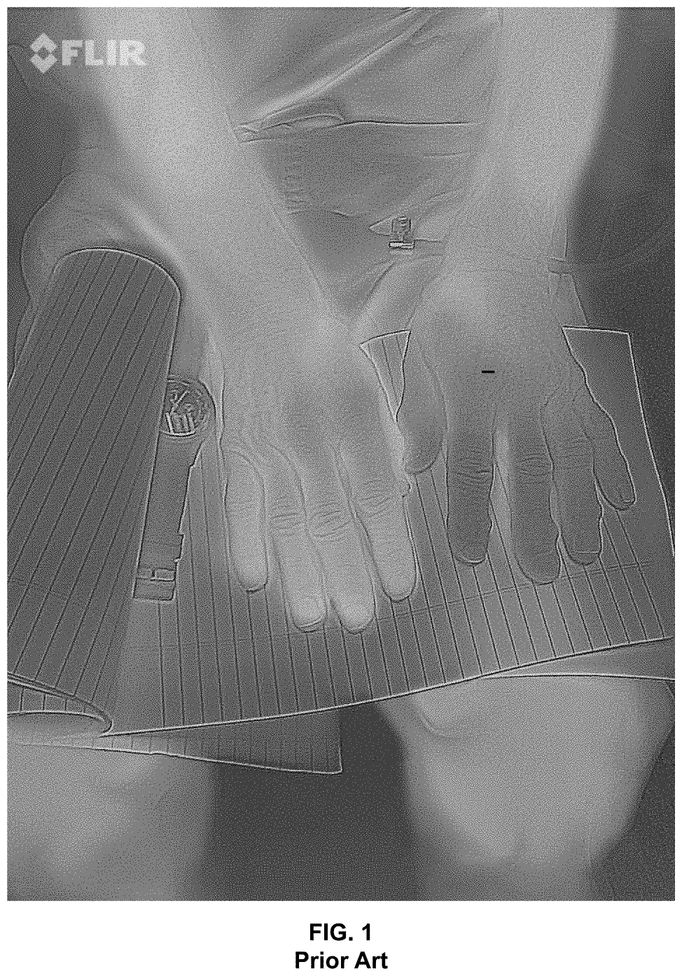

### DETAILED DESCRIPTION OF THE EMBODIMENTS

As required, detailed embodiments of the systems, apparatuses, and methods are disclosed herein; however, it is to be understood that the disclosed embodiments are merely exemplary of the systems, apparatuses, and methods, which can be embodied in various forms. Therefore, specific structural and functional details disclosed herein are not to be interpreted as limiting, but merely as a basis for the claims and as a representative basis for teaching one skilled in the art to variously employ the systems, apparatuses, and methods in virtually any appropriately detailed structure. Further, the terms and phrases used herein are not intended to be limiting; but rather, to provide an understandable description of the systems, apparatuses, and methods. While the specification concludes with claims defining the features of the systems, apparatuses, and methods that are regarded as novel, it is believed that the systems, apparatuses, and methods will be better understood from a consideration of the following description in conjunction with the drawing figures, in which like reference numerals are carried forward.

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Alternate embodiments may be devised without departing from the spirit or the scope of the invention. Additionally, well-known elements of exemplary embodiments of the systems, apparatuses, and methods will not be described in detail or will be omitted so as not to obscure the relevant details of the systems, apparatuses, and methods.

Before the systems, apparatuses, and methods are disclosed and described, it is to be understood that the terminology used herein is for the purpose of describing particular embodiments only and is not intended to be limiting. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact (e.g., directly coupled). However, “coupled” may also mean that two or more elements are not in direct contact with each other, but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, top/bottom, and proximal/distal. Such descriptions are merely used to facilitate the discussion and are not intended to restrict the application of disclosed embodiments. Various operations may be described as multiple discrete operations in turn, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means that, when comparing various parts to one another, the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

It will be appreciated that embodiments of the systems, apparatuses, and methods described herein may be comprised of one or more conventional processors and unique stored program instructions that control the one or more processors to implement, in conjunction with certain non-processor circuits and other elements, some, most, or all of the functions of the systems, apparatuses, and methods described herein. The non-processor circuits may include, but are not limited to, signal drivers, clock circuits, power source circuits, and user input and output elements. Alternatively, some or all functions could be implemented by a state machine that has no stored program instructions, or in one or more application specific integrated circuits (ASICs) or field-programmable gate arrays (FPGA), in which each function or some combinations of certain of the functions are implemented as custom logic. Of course, a combination of these approaches could also be used. Thus, methods and means for these functions have been described herein.

The terms “program,” “software,” “software application,” and the like as used herein, are defined as a sequence of instructions designed for execution on a computer system or programmable device. A “program,” “software,” “application,” “computer program,” or “software application” may include a subroutine, a function, a procedure, an object method, an object implementation, an executable application, an applet, a servlet, a source code, an object code, any computer language logic, a shared library/dynamic load library and/or other sequence of instructions designed for execution on a computer system.

Herein various embodiments of the systems, apparatuses, and methods are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

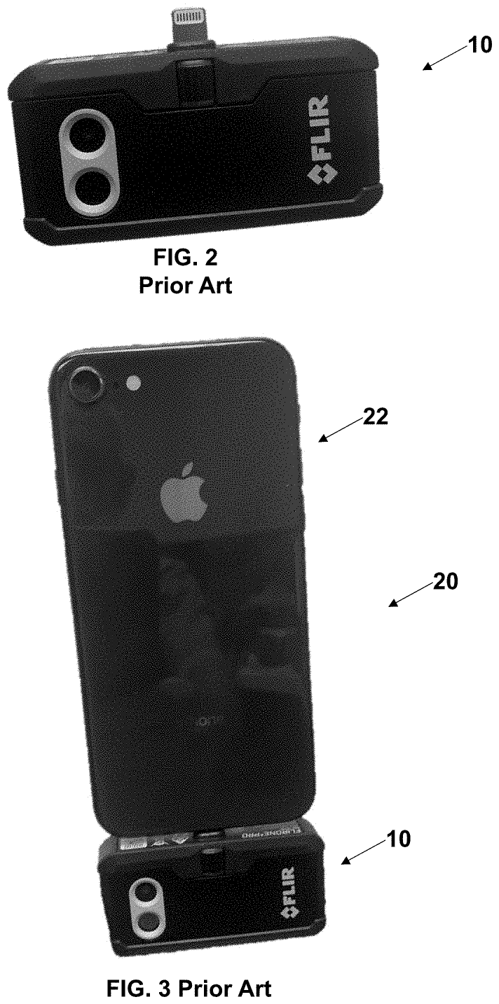

FIG. 2 illustrates an exemplary embodiment of the systems, processes, and methods utilizing the infrared spectrum of a prior art forward-looking infrared camera 10 made by FLIR® and sold under the name “Flir ONE PRO-iOS Pro-Grade Thermal Camera for Smartphones”. The hardware and software taking the thermal images is referred to herein collectively as a thermal imager or a thermal imager of the camera 10. This is only one exemplary embodiment of a camera 10 as various other imaging devices can perform the same or similar data acquisition to determine normal/abnormal perfusion. FIG. 3 illustrates an exemplary embodiment of a transportable camera system 20 with the camera 10 communicatively connected to a computer processor 22, here in the form of a smartphone and, together in an exemplary embodiment, forming a transportable infrared (IR) camera system. This is only one exemplary embodiment of the transportable camera system 20 as various other portable and transportable processing devices can perform the same or similar data acquisition, thermal or otherwise. For example, a portable processing device can include a tablet (e.g., an iPad) and a transportable processing device can include a mobile computer, the latter being attached, for example, to an arm that can be secured to a patient's bed or on a cart and able to be rolled up to a patient as desired.

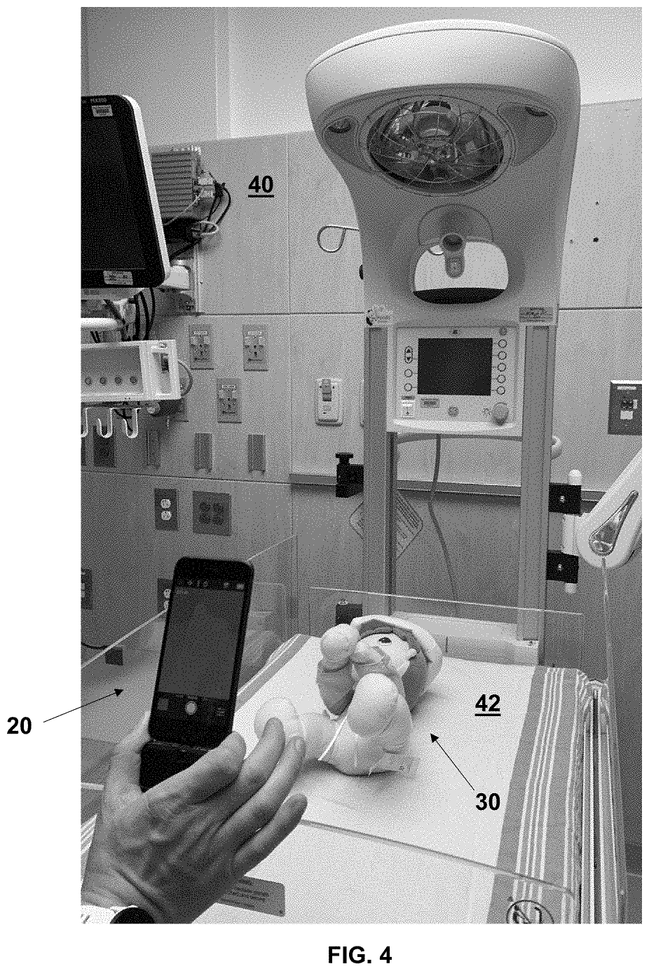

Described now are exemplary embodiments. Referring now to the figures of the drawings in detail and first, particularly to FIG. 4, there is shown an exemplary embodiment of a transportable camera system 20 that is based on thermal imaging. Before the advent of smartphones and to the miniaturization of forward-looking infrared cameras, components that could take IR images of a person included large and expensive desktop computers and equally large and expensive IR cameras. If the user wanted to detect and analyze those images, large computer systems were required. These systems were too bulky and expensive to be mounted at each patient's bed. Now, with smartphones being ubiquitous and with IR cameras 10 being so compact and inexpensive (and other cameras in a different electromagnetic spectrum), it becomes possible to mount a complete, transportable camera system 20 at each patient's bedside. Further, advances in digital signal processing, as it applies to the electronic images that are output by such cameras 10, allow the transportable camera system 20 to perform real-time instantaneous monitoring and assessment of a patient in a manner that was not heretofore possible. In use, the transportable camera system 20 in the infrared embodiment takes thermal images (including both stills and video), either manually, periodically, or continually, and with an app or another form of software, stores the thermal images locally and/or remotely. Where the software is resident on the transportable camera system 20, it can be an app, a program, or firmware, or a combination of any of these; where the software is resident on a server or another separate computer accessible through a communications link, for example, through the internet cloud, it can be an app, a program, or firmware, or a combination of any of these.

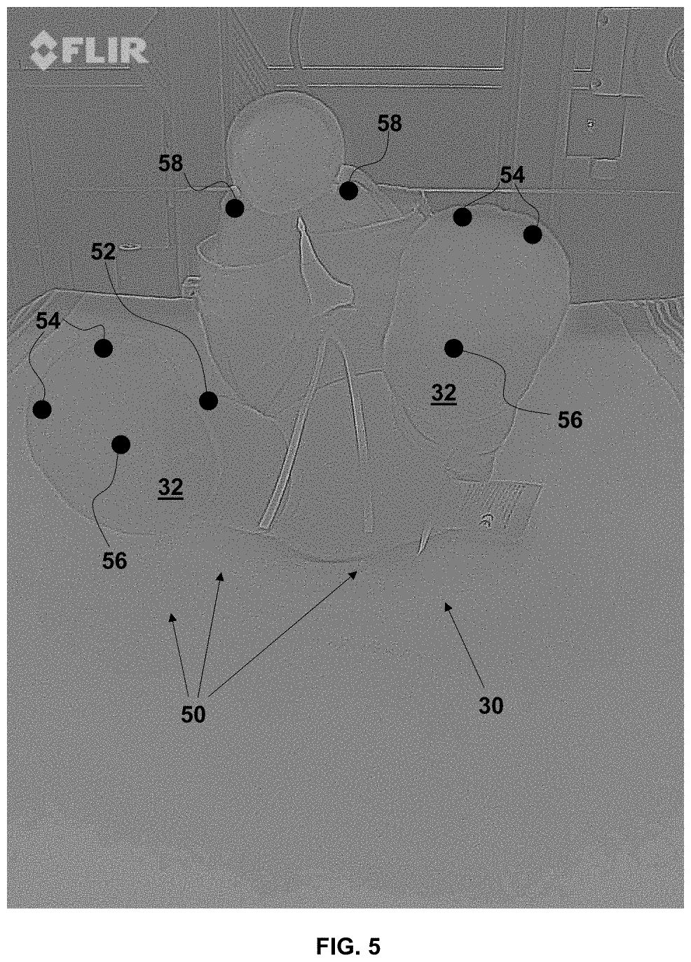

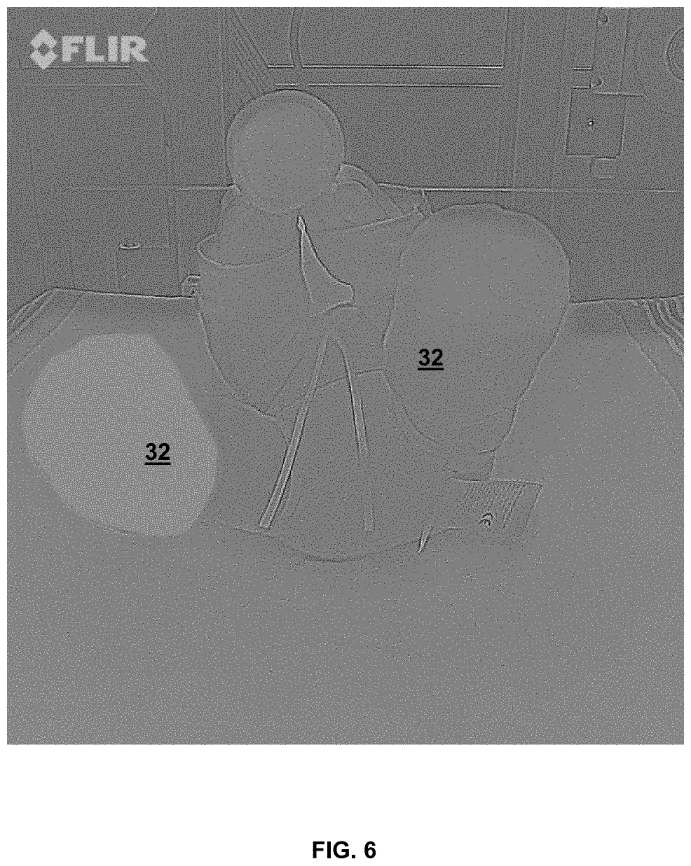

In an exemplary process for carrying out a monitoring and analysis method in a neonatal intensive care unit (NICU) 40, a patient 30 is admitted and is placed in a NICU bed 42, which can be referred to as a patient workspace. To detect abnormal perfusion of the patient's legs (for example with a thermal imaging camera), one or more of the transportable IR camera systems 20 are placed at a foot of the bed 42 to observe both legs of the patient 30. In an exemplary embodiment, easy-to-detect IR markers 50 are placed on one or more extremities as indicators of the anatomy upon which the transportable camera system 20 will focus. Exemplary registering locations for these markers 50 include knee markers 52, one or more toe markers 54 on opposite feet, one or more sole markers 56 on the soles of the patient's feet 32, and/or one or more hand markers 58, examples of each are shown in FIG. 5. Software in the transportable camera system 20 is programmed to make a number of determinations based on at least one image received (photo and/or video). The determinations can be periodic, at a user's command, or in real time, for example. Various exemplary determinations by the software include, but are not limited to:
defining bilateral temperature of the patient's skin (e.g., difference in temperature of the patient's hands and/or fingers and/or feet and/or toes); and/or
defining the location of temperature on one or more extremities and/or locations on those extremities; and/or
defining the temperature of one extremity compared to the other extremity; and/or
defining trends of these detected temperatures.
   The thermal image of FIG. 6, for example, shows the two feet 32 of an infant patient having vastly different temperature readings. With such measurements, analysis in terms of comparing data extracted from the image (or these images) becomes possible. For example, software (which can include artificial intelligence or expert systems) analyzes the detected/measured data and determines if the instantaneous reading and/or trend is/are within a previously determined normal variation (compared to a predefined temperature or a core temperature in the EMR) or is indicating an alarming sign of bilateral or unilateral extremity altered perfusion. In the example of FIG. 6, the temperature comparison of the soles of the two feet 32 will indicate instantaneous decreased temperature as compared to the other foot 32 and unilateral decreasing temperature over time, which indicates a serious condition of arterial or venous occlusion, e.g., from an embolus or vascular trauma. In an exemplary embodiment, all data is recorded in the EMR and alarms are triggered as programmed by the system, the user, and/or the facility.

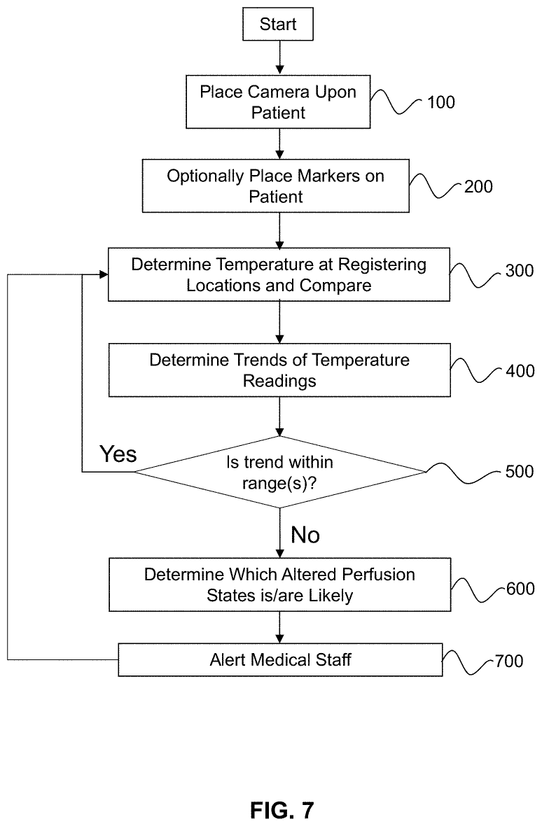

An exemplary process for carrying out real-time, instantaneous monitoring and assessment of a patient 30 admitted to the ICU, for example, is described with regard to FIG. 7. In Step 100, one or more transportable camera systems 20 are placed adjacent a patient's bed (e.g., at a foot of the bed to observe both feet and/or legs of the patient 30). In the exemplary embodiment, the camera systems 20 are thermal imaging cameras. If desired, in Step 200, markers 50 are placed on the patient as indicators of anatomy (e.g., knees 52, shins 52/54/56, toes 54, soles 56); these are the registering locations. The camera(s) 10 and the associated processing device(s) 22 of the transportable camera system(s) 20 are programmed to determine the bilateral temperature of the patient's skin (e.g., at the registering location(s) and/or adjacent the marker location(s)) and temperature is taken from the images in Step 300. This allows the transportable camera system 20 to determine the location of temperature on one or more extremities and, therefore, to determine the temperature of one extremity and compare it to the temperature of the other extremity (or to a predefined temperature value) and, in storing this data (permanently or temporarily), to also determine trends of the detected temperature(s), which occurs in Step 400. Software and/or firmware (which can include AI or expert systems) analyses the detected/measured and digitally processed data and determines if the trend is within previously determined variations (e.g., within a defined “normal” state and/or rate of change). Any detection outside the expected or predefined variations, therefore, indicate various altered pathological perfusion conditions, which indication is performed in Step 500. Particular data and/or data trends may indicate (through pre-defined stored conditional data) one or more particular pathological states. Such trends might, for example, include abrupt decrease in perfusion in one extremity or more, which indicates the possibility of acute arterial occlusion. Another trend may indicate decreasing perfusion bilaterally over time, which indicates decreasing cardiac output. Alternatively or additionally, a determined trend may indicate bilateral increase in perfusion, which indicates loss of vascular integrity and impending sepsis. With this data and/or trend(s), in step 600, the software determines what kind of medically different causes of temperature variation is occurring presently, for example:
increase in temperature bilaterally (impeding sepsis); or
bilateral decreasing temperature (decreasing cardiac output); or
unilateral decreasing temperature (arterial of venous occlusion, from an embolus or vascular trauma).
   One exemplary process includes the software comparing calculated or determined data to the patient's core temperature, which the medical staff 80 had stored or periodically stored/stores in the EMR. In another exemplary embodiment, the software determines if a trend is an alarming sign of bilateral or unilateral extremity altered perfusion. If any triggering event occurs, in Step 700, the transportable camera system 20 alerts medical staff 80 in real-time. In an exemplary embodiment, all thermal data is recorded in the EMR and alarms are triggered as programmed or desired. The process continues in real-time as long as the medical staff 80 desire.

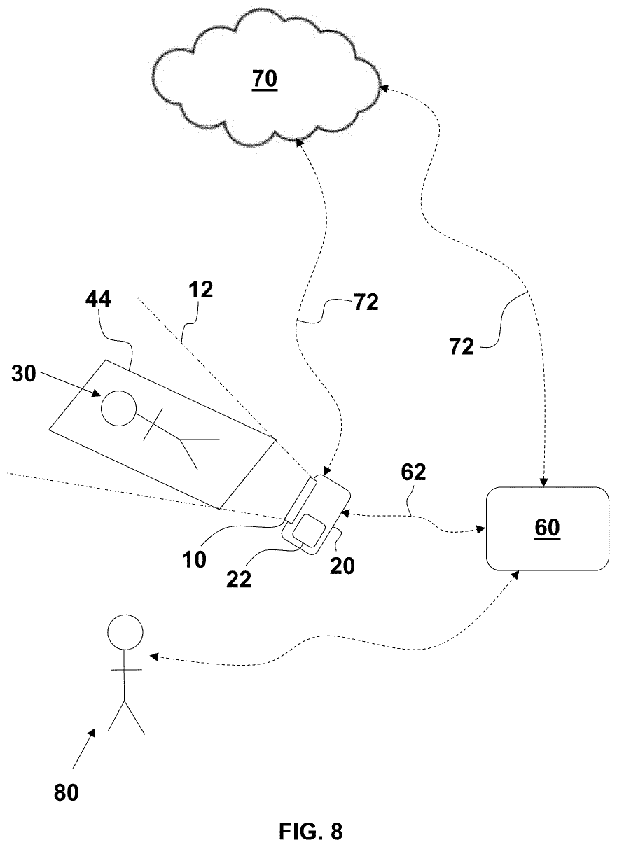

FIG. 8 illustrates the movement of information or data collected by the transportable camera system 20 in the embodiment using electromagnetic imaging techniques within any part(s) of the spectrum. The patient 30 is in the patient workspace 44 and the transportable camera system 20 is located to point the camera 10 at the patient workspace 44 so that the patient 30 remains within the viewable area 12 of the camera 10. The camera 10 periodically and/or continually takes photos and/or video and communicates this image/these images to the processor 22 of the transportable camera system 20. Either the transportable camera system 20 (using the processor 22 onboard) and/or software in the cloud 70 (using a second processor off-board and associated with the transportable camera system 20) analyzes the data and determines and/or calculates corresponding extremity temperature(s) and/or trend(s) (e.g., for each photo or frame of a video). An exemplary association of the transportable camera system 20 to the second separate processor is through a communications link 72, which can include communication through the internet cloud 70. In such an exemplary embodiment, the transportable camera system 20 communicates (wirelessly or wired) to the EMR 60 either in a direct link 62 (e.g., Bluetooth®) or through the internet cloud 70. Then, either the system 20 (using the processor 22) or software in the cloud 70 associated with the system 20 determines the status of that data as being normal (previously determined normal variation) or as requiring attention or alarm. The EMR 60 communicates with medical staff 80 so that appropriate action can be taken as desired. In FIG. 8, the EMR 60 is shown as separate from the transportable camera system 20. In an exemplary embodiment, the EMR 60 is integrated within the transportable camera system 20.

It is noted that various individual features of the inventive processes and systems may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all of the other exemplary embodiments described herein and in any combination or grouping or arrangement. In particular, use of a single reference numeral herein to illustrate, define, or describe a particular feature does not mean that the feature cannot be associated or equated to another feature in another drawing figure or description. Further, where two or more reference numerals are used in the figures or in the drawings, this should not be construed as being limited to only those embodiments or features, they are equally applicable to similar features or not a reference numeral is used or another reference numeral is omitted.

The foregoing description and accompanying drawings illustrate the principles, exemplary embodiments, and modes of operation of the systems, apparatuses, and methods. However, the systems, apparatuses, and methods should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the systems, apparatuses, and methods as defined by the following claims.

What is claimed is:

1. A thermal imaging extremity altered perfusion detector system, comprising:
a computer processor configured to receive and to at least temporarily store thermal images; and
a thermal imaging camera communicatively coupled to the computer processor and configured to take at least one of photograph and video thermal images and output the thermal images to the computer processor, the thermal imaging camera:
configured to be secured adjacent a patient workspace that is shaped to contain a patient; and
pointing the thermal imaging camera at the patient workspace such that, responsive to taking at least one thermal image, the at least one thermal image contains at least a portion of the patient who is placed within the patient workspace;
wherein the computer processor is configured to analyze the at least one thermal image and determine from the at least one thermal image a difference in thermal states indicating altered perfusion in at least one of the extremities of the patient.

2. The system according to claim 1, wherein the computer processor is one of a smart phone and a tablet.

3. The system according to claim 1, wherein the thermal imaging camera is a forward-looking infrared camera.

4. The system according to claim 2, wherein the thermal imaging camera is a forward-looking infrared camera and, together with the computer processor, form a transportable IR camera system configured to acquire and to at least temporarily store the at least one thermal image.

5. The system according to claim 4, wherein:
the at least one thermal image comprises an image of at least a portion of the patient; and
the transportable IR camera system is configured to analyze the at least one thermal image and thereby determine altered perfusion of at least one of the extremities of the patient.

6. The system according to claim 5, wherein the transportable IR camera system is configured to perform real-time instantaneous monitoring of the at least one of the extremities.

7. The system according to claim 5, wherein the at least one thermal image is a video.

8. The system according to claim 5, wherein the transportable IR camera system is configured to acquire the at least one thermal image at least one of manually, periodically, and continually.

9. The system according to claim 5, wherein the transportable IR camera system is configured to store the at least one thermal image at least one of locally and remotely.

10. The system according to claim 1, wherein the thermal imaging camera comprises a thermal imager configured to obtain thermal images in the form of at least one of stills and video.

11. The system according to claim 10, wherein the computer processor comprises software and is configured to analyze the at least one of stills and video and to determine an altered perfusion state of the patient with the software.

12. The system according to claim 11, wherein the computer processor is configured to analyze the at least one of stills and video in real-time.

13. The system according to claim 1, wherein the computer processor is a first computer processor, and which further comprises a second computer processor separate from the first computer processor and communicatively connected to the first computer processor through at least one communications link, the second computer processor being configured to analyze the at least one of stills and video, to determine an altered perfusion state of the patient, and to output the altered perfusion state.

14. The system according to claim 13, wherein the second computer processor communicates through the at least one communications link an indication of the altered perfusion state of the patient.

15. The system according to claim 12, wherein the at least one communications link comprises the internet cloud and the second computer processor comprises at least one of a mainframe, a server, a desktop, and a laptop.

16. The system according to claim 1, wherein the computer processor analyzes the difference in thermal states to indicate at least one of impeding sepsis, bilateral decreasing temperature, and unilateral decreasing temperature.

17. The system according to claim 14, wherein the indication of the altered perfusion state includes at least one of impeding sepsis, bilateral decreasing temperature, and unilateral decreasing temperature.

18. The system according to claim 1, further comprising an electronic medical record of the patient, the computer processor being configured to integrate the difference in thermal states into the electronic medical record in real time and to drive real time alarms for appropriate altered perfusion states.

19. The system according to claim 1, further comprising markers configured to be placed on the extremities of the patient as registering locations, the computer processor being configured to determine the difference in thermal states at least one of at and adjacent the markers.

20. The system according to claim 1, wherein the computer processor is configured:
to determine data comprising at least one of a temperature of the patient's skin, a location of temperature on one or more extremities, a temperature of one extremity compared to another extremity, and trends of the detected temperatures; and
to analyze the determined data and to determine if a trend the determined data is within a previously determined normal variation.

21. A thermal imaging extremity altered perfusion detector system, comprising:
a smartphone configured to receive and to at least temporarily store thermal images;
markers configured to be placed on the extremities of the patient as registering locations; and
a thermal imaging camera communicatively coupled to the computer processor and configured to take at least one of photograph and video thermal images and output the thermal images to the smartphone, the thermal imaging camera:
configured to be secured adjacent a patient workspace that is shaped to contain a patient; and
pointing the thermal imaging camera at the patient workspace such that, responsive to taking at least one thermal image, the at least one thermal image contains at least a portion of the patient who is placed within the patient workspace;
wherein the smartphone is configured:
to analyze the at least one thermal image and determine from the at least one thermal image a difference in thermal states at least one of at and adjacent the markers indicating altered perfusion in at least one of the extremities of the patient in real-time;
based upon the determined difference, to communicate an indication of the altered perfusion state of the patient indicating at least one of impeding sepsis, bilateral decreasing temperature, and unilateral decreasing temperature; and
to integrate the altered perfusion state into an electronic medical record of the patient.

22. An imaging extremity altered perfusion detector system, comprising:
a computer processor configured to receive and to at least temporarily store electronic images; and
an imaging camera communicatively coupled to the computer processor and configured to take at least one of photograph and video images and output the images to the computer processor, the imaging camera:
configured to be secured adjacent a patient workspace that is shaped to contain a patient; and
pointing the imaging camera at the patient workspace such that, responsive to taking at least one image, the at least one image contains at least a portion of the patient who is placed within the patient workspace;
wherein the computer processor is configured to analyze the at least one image and determine from the at least one image a difference in states indicating altered perfusion in at least one of the extremities of the patient.

23. The system according to claim 22, wherein the at least one of photograph and video images are at least one of radio, microwave, thermal, visible, ultraviolet, sonography, computed tomography, magnetic resonance imaging, x-ray, and gamma ray images.
